(19) United States
(12) Patent Application Publication
(10) Pub. No.: US 2016/0120509 A1
(43) Pub. Date: May 5, 2016

(54) HYPODERMIC NEEDLE WITH ENHANCED ULTRASOUND SIGNATURE FOR CAROTID ARTERY STENTING

(71) Applicant: Mubin I. Syed, Springfield, OH (US)

(72) Inventors: Mubin I. Syed, Springfield, OH (US); Michael Hogendijk, Santa Rosa, CA (US); Reuvan Marko, Netanya (IL)

(21) Appl. No.: 14/926,659

(22) Filed: Oct. 29, 2015

Related U.S. Application Data
(60) Provisional application No. 62/072,497, filed on Oct. 30, 2014.

(51) Int. Cl. A61B 8/08; A61B 8/00

(52) U.S. Cl. CPC A61B 8/481; A61B 8/0841; A61B 8/4483; A61B 2017/3413

(57) ABSTRACT

A hypodermic needle outer surface deflects ultrasound signals hitting the surface. The reflected signals depend on the surface which the ultrasound signal hits. According to the invention the outer surface of the needle, beginning at the tip or cutting edge of the needle featuring any one or more of: a) dimples having one or more distinct patterns; b) pits, randomly scattered; and, c) double helix, i.e., two grooves starting at an angle apart, preferably 180 degrees, and spiraling backwards in a helical fashion from the tip. By periodically repeating different pattern(s) for a predetermined length of the needle the position of the needle can be accurately identified using ultrasound imaging. The needle, in different lengths and gauges, can be used to facilitate stenting of the carotid artery as well as other ultrasound guided procedures such as biopsies, drainages/aspirations, solid organ tumor ablations and vascular access in general.

### CROSS-REFERENCE TO RELATED APPLICATIONS

This patent application claims priority to U.S. provisional patent application 62/072,497, filed Oct. 30, 2014, the disclosure of which is herein incorporated by reference in their entirety.

### BACKGROUND

1. Field

The invention relates to medical devices inserted into bodily arteries and or other organs, and more particularly to a needle used therein, the needle having echogenic properties typically for use in conjunction with an ultrasound generator.

2. Related Art

Plaque buildup or lesions in a patient's carotid artery, which is the main artery from the aorta/heart to the brain, are routinely stented. This is done by using a metal scaffold that opens the artery by pushing the plaque that has gathered therein out of the way. The juncture of the aorta artery to the carotid artery can be difficult to pass through due to plaque buildup and/or anatomical reasons. Typically, the procedure is done under fluoroscopy which is an X-ray machine that allows a practitioner to view the procedure in real-time. Access is gained to the patient's carotid artery by first passing a guidewire from the patient's right or left common femoral artery, i.e., the main groin artery, up the patient's aorta artery, into the carotid artery, and then across the legion, i.e., the plaque build-up area. A guide catheter may then be inserted proximal to the lesion allowing to inject a contrasting material through it and see the legion as it is being evaluated as well as stented. Secondly a stent delivery catheter is tracked over the guidewire and through the guide catheter and across the lesion. Thereafter the stent is deployed and the guidewire, embolic protection device, and catheter removed.

A fluoroscope is used in the process it has an X-ray source and a fluorescent screen or another kind of sensor, for example Intra Vascular Ultrasound (IVUS). Using fluoroscopy typically exposes the patient to a fair amount of undesirable radiation as that is a function of the length of the procedure. The longer the exposure the larger the radiation dose the patient is exposed to and therefore the increased risk.

Accordingly, what is needed is a solution that would reduce the risk of exposure to an undesirable dosage of X-ray when performing the placement of a stent when treating plaque buildup or lesions in a patient's carotid artery involving a hostile aortic arch or torturous anatomy preferably using ultrasound imaging as it has no radiation in its operation.

### SUMMARY

The following summary of the invention is included in order to provide a basic understanding of some aspects and features of the invention. This summary is not an extensive overview of the invention and as such it is not intended to particularly identify key or critical elements of the invention or to delineate the scope of the invention. Its sole purpose is to present some concepts of the invention in a simplified form as a prelude to the more detailed description that is presented below.

In accordance with one aspect of the invention, a system is disclosed that includes a hypodermic needle body comprising a proximal end and a distal end and a plurality of surface artifacts in the body; and an ultrasound transducer configured to emit ultrasound waveforms, wherein the surface artifacts are configured to reflect the ultrasound waveforms.

The surface artifacts may be selected from the group consisting of a double helix groove, dimples, pitts, and circular grooves.

In accordance with another aspect of the invention, a hypodermic needle is disclosed that includes a needle tube main body comprising an edge at a tip end of the hypodermic needle, the needle tube having an outer surface; at least a first helix groove on the outer surface of the needle tube beginning at a proximity of the tip end and spiraling at a first pitch; and at least a second helix groove on the outer surface of the needle tube beginning at a proximity of the tip end and spiraling at a second pitch; wherein the starting point of the first helix with respect of the tip end and the starting point of the second helix with respect of the tip end is different.

The first starting point and the second starting point may be opposite each other.

The first pitch and the second pitch may be equal.

The second helix groove may spiral counterclockwise.

The first helix groove and the second helix groove may cross each other at at least one crossing point. The at least one crossing point may be periodic.

In accordance with a further aspect of the invention, a hypodermic needle is provided for traceability at insertion using ultrasound that includes a needle tube main body comprising an edge at a tip end of the hypodermic needle, the needle tube having an outer surface; and a plurality of bands on the outer surface of the needle tube each of the plurality of bands having a length and each having a pattern that is at least different from an adjacent band; wherein each pattern used for any band of the plurality of bands has a unique ultrasound deflection.

The hypodermic needle may further include a patternless band, the patternless band positioned between two adjacent patterned bands of the plurality of bands.

The hypodermic needle may further include a plurality of patternless bands, each of the plurality of patternless bands positioned between two adjacent patterned bands of the plurality of bands.

The length of each of the plurality of bands may be the same.

Each of the plurality of bands may have a unique pattern. The pattern may be selected from the group consisting of at least a double helix grooves, dimples, pitts, and circular grooves.

A first helix of the at least a double helix may have a first pitch and a second helix of the at least a double helix may have a second pitch. The first helix may spiral clockwise and the second helix may spiral counterclockwise.

The dimples may be positioned randomly within a band. The dimples may be positioned periodically within a band. The dimples may be of: equal size or random size.

The pitts may be positioned randomly within a band. Tthe pitts may be positioned periodically within a band. The pitts may be of: equal size or random size.

### BRIEF DESCRIPTION OF THE DRAWINGS

The accompanying drawings, which are incorporated into and constitute a part of this specification, illustrate one or more examples of embodiments and, together with the description of example embodiments, serve to explain the principles and implementations of the embodiments.

FIG. 1 is a perspective view of a tip of a needle having improved echogenicity in accordance with one embodiment of the invention.

FIG. 2 is a perspective view of a tip of a needle having improved echogenicity in accordance with one embodiment of the invention.

FIG. 3 is a schematic diagram illustrating the use of a needle having improved echogenicity in use with an ultrasound system in accordance with one embodiment of the invention.

### DETAILED DESCRIPTION

Embodiments of the invention are directed to a hypodermic needle, which includes an outer surface that is capable of deflecting ultrasound signals that hit its surface. The reflected signals depend on the surface which the ultrasound signal hits. The outer surface of the needle, beginning at the tip or cutting edge of the needle, may featuring any one or more of: a) dimples having one or more distinct patterns; b) pits, randomly scattered; and, c) double helix, i.e., two grooves starting at an angle apart, preferably 180 degrees, and spiraling backwards in a helical fashion from the tip. By periodically repeating different pattern(s) for a predetermined length of the needle, the position of the needle can be accurately identified using ultrasound imaging. The needle, in different lengths and gauges, can be used to facilitate stenting of the carotid artery as well as other ultrasound guided procedures such as biopsies, drainages/aspirations, solid organ tumor ablations and vascular access in general.

The use of a needle according to the various embodiments discussed herein allows for the use of ultrasound to perform stenting and other procedures, avoiding the need to expose the patient to an unnecessary dosage of X-ray radiation. For example, but not by way of limitation, the needle may be used in the case of treatment of plaque buildup or lesions in a patient's carotid artery involving a hostile aortic arch or torturous anatomy. In such cases, the known systems and methods would require excess exposure to fluoroscopy which may be avoided by using the improved echogenic needle disclosed herein which is ultrasound guided through a supplementary superficial temporal artery access. To successfully meet the requirement to be able to operate under ultrasound in a precise manner, the echogenicity of the needle used is increased. Embodiments of the invention are directed to adding a feature or features to the outer surface of the needle beginning at the tip or cutting edge of the needle and extending back towards the needle hub. In one embodiment, such extension continues for approximately 2 to 5 millimeters.

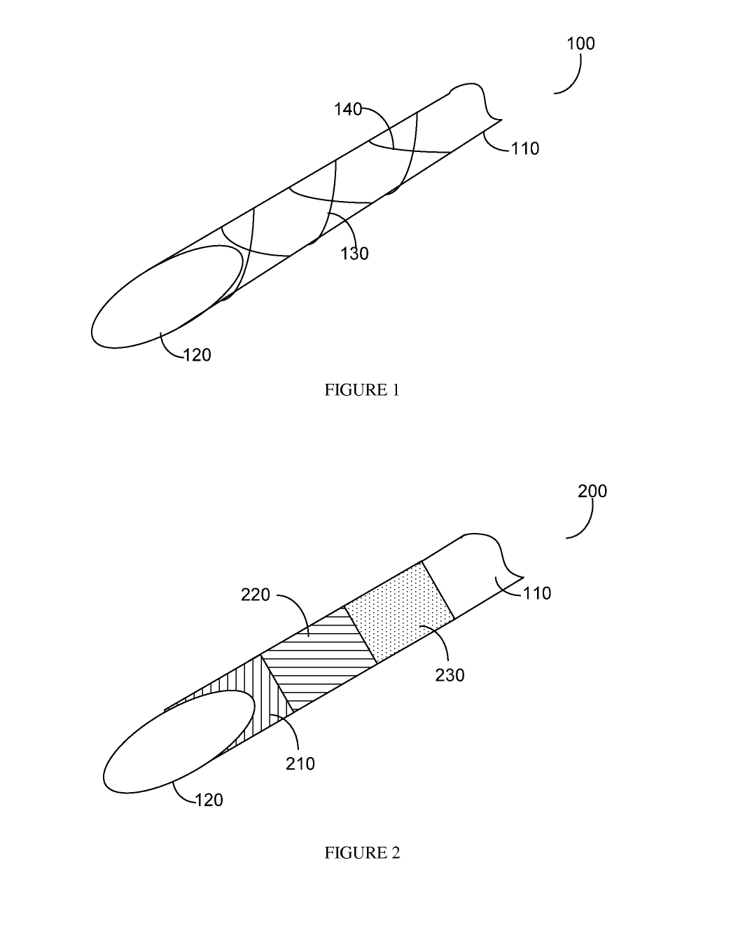

Reference is now made to FIG. 1 which depicts an exemplary and non-limiting portion 100 of a needle 110 having improved echogenicity according to a first embodiment. In FIG. 1, the improved echogenicity of the needle 110 is provided by using a double helix, having two grooves (groove 130 and groove 140) beginning 180 degrees form each other and spiraling backwards from the tip 120 of the needle in a helical fashion. Each helix has a pitch that may be in the range extending from an almost flat pitch, for example 1°, to an almost perpendicular pitch, for example 89°. In one embodiment, the first helix 130 turns clockwise while the second helix turns counterclockwise 140. In another embodiment (not shown), the first helix may spiral at a pitch that is different from the pitch of a second helix.

When an ultrasound wave hits the needle area where the grooves 130 and 140 exist, there will be a distinct deflection of the wave to the ultrasound transducer (not shown) which can be used to provide to the practitioner, after appropriate processing, an image of the needle 110 as it is inserted into the artery. While two grooves are shown herein, one of ordinary skill in the art would readily appreciate that other embodiments are also possible that include a triple helix comprising of three grooves, four grooves, and so on and so forth. If symmetry is maintained than in the case of a triple helix the grooves will have a 120 degree offset of each other. However, this should not be viewed as a limitation and embodiments where an asymmetrical offset is used are specifically within the scope of the invention. Depth of a helix groove may be of any depth between 0.1-90% of the thickness of the cross section of the needle.

FIG. 2 depicts an exemplary and non-limiting portion 200 of a needle 110 having improved echogenicity according to one embodiment of the invention. The distance going back from the tip 120 is divided into multiple sections, for example, section 210, section 220 and section 230. Each section 210, 220, 230 has unique pattern on its surface of the needle 110. As a result, when an ultrasound transducer (not shown) emits an ultrasound wave, each of the sections 210, 220 and 230 will have a distinct reflection pattern. The returned ultrasound waves may be captured and processed to provide an accurate image that also provides a distance metric to the practitioner. For example, in one embodiment, each of the sections 210, 220 and 230 may be one millimeter long. In one embodiment, section 210 could have a pattern of a double helix, section 220 a pattern of a triple helix, and section 230 a pattern of a quadruple helix. One of ordinary skill in the art would readily appreciate that the number of different patterns may be two or more, and that further a sequence of patterns may also be created, for example, the sequence may begin with the sequence 210, 220 and 230, and then followed, continuing backwards from the tip 120 on the needle 110, with sections having first the pattern 220 and then the pattern 210, providing a section sequence of 210, 220, 230, 220, 210 (the sequence is not shown). It will be appreciated that any number of sections may be used, and any number of sequences of sections may be used. If a coarser granularity is desired than the section length may be increased, for example to 1.5 or 2 millimeters. Conversely, if a finer granularity is required, a section length may be reduced, for example, to 0.5 a millimeter. It will be appreciated that the lengths may be any value or range of values between 0.5 and 2 mm; it will also be appreciated that the lengths may be less than 0.5 mm or more than 2 mm.

It will be appreciated that patterns other than the likes of a double helix may be used to distinguish one section, for example section 210, from another section, for example section 220. For example, in one embodiment a pattern of dimples, organized in a pattern and/or having particular diameters, sizes and/or shapes may be used. The pattern may be random or repetitive. Each pattern, according to the invention, is designed to provide a different deflection of the ultrasound waves thereby make it uniquely identifiable. In another embodiment, a pattern of pitts, organized in a pattern and/or having particular diameters, sizes and/or shapes may be used. The pattern may be random or repetitive. Each pattern, according to the invention, is designed to provide a different deflection of the ultrasound waves thereby make it uniquely identifiable. In yet another embodiment each section comprises circular groove around the needle 110 at a particular section. Sections can differ from one another by the density of such grooves. One of ordinary skill in the art would readily appreciate that any two patterns can be separated by a section that has no pattern, spacing the rings, for example and without limitation, every 2 millimeters. In yet another embodiment the lengths of each of the sections, for example, sections 210 and section 220, may be different. Moreover, one of ordinary skill-in-the-art would readily realize that the needle may be implemented in a variety of sizes in length and diameter. The depth of the grooves, pitts, dimples and the like, may be any value or range of values between 0.1-90% of the thickness of the cross section of the needle.

One of ordinary skill in the art would further appreciate that a practitioner using a needle having enhanced echogenic properties according to the invention, could, under the use of ultrasound perform a procedure wherein stenting can be performed in cases, such as but not limited to, difficult carotid stenting, biopsy procedures or any other procedure wherein the need for an ultrasound needle with improved echogenicity is required. In the case of carotid stenting a guidewire is passed through a needle placed into the superficial temporal artery allowing the actual stenting itself to be performed by using ultrasound. In the case of carotid stenting an ultrasound guided access to the superficial temporal artery branch of the external carotid artery is used to facilitate internal carotid stenting in the case of a tortuous anatomy (hostile aortic arch). The ultrasound guided access to the superficial temporal artery serves as an anchor point, that is in addition to the groin access, which enables to stabilize the guidewires so that the stent delivery system and guidewires in the internal carotid artery do not get pushed out of the internal carotid artery during the procedure. In one embodiment while part of the medical procedure is performed under fluoroscopy, the patient is exposed, according to the disclosed medical procedure to a reduced dosage of X-ray radiation.

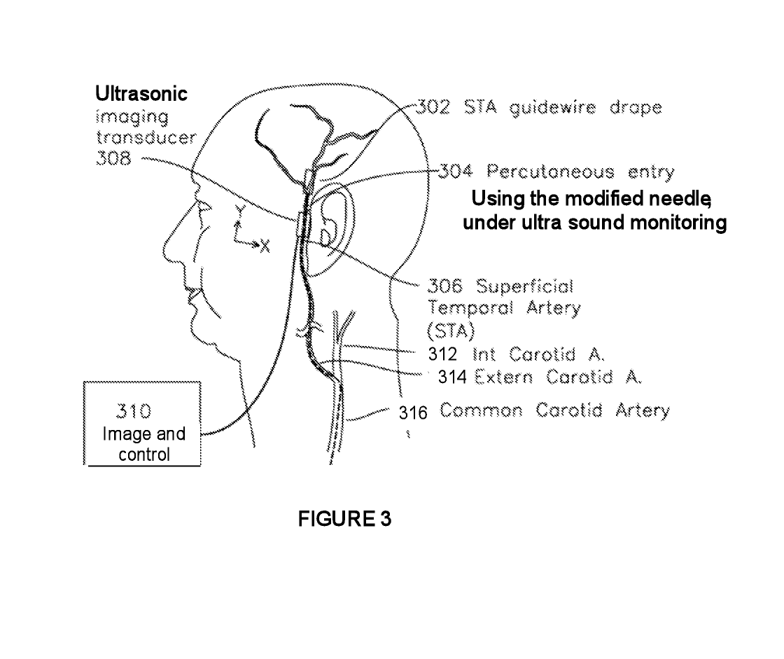

FIG. 3 illustrates an exemplary use of the hypodermic needle as disclosed herein. FIG. 3 illustrates the use of the hypodermic needle for percuatenous entry into a superficial temporal artery. It will be appreciated that FIG. 3 illustrates one exemplary use and that other exemplary uses are contemplated by the invention. For example, as described above, the hypodermic needle may be used for difficult carotid stenting, biopsy procedures, drainages/aspirations, solid organ tumor ablations, vascular access or any other procedure wherein the need for an ultrasound needle with improved echogenicity is required.

In FIG. 3, the common carotid artery 316, internal carotid artery 213, external carotid artery 314 and superficial temporal artery (STA) 306 are illustrated. In use, a STA guidewire 302 may be delivered percutaneously to the STA 306 through the common carotid artery 316 and external carotid artery 314 with the assistance of the hypodermic needle having surface artifacts, as described herein with reference to, for example, FIGS. 1 and 2. For example, the surface artifacts may be in the form of a double helix formed in the body of the hypodermic needle. Alternatively, or in addition to the double helix, pitts, dimples, grooves and the like may be used to provide echogenicity to the hypodermic needle. As shown in FIG. 3, the hypodermic needle is also entered percutaneously at percutaneous entry site 304. An imaging transducer 308 is used to generate ultrasound waveforms that are directed toward the hypodermic needle. The surface artifacts on the needle reflect the ultrasound waveforms, which are measurable by an image and image control console 310 using known techniques and systems. For example, General Electric's transducers and LOGIQ imaging system can be used as the transducer 308 and the image and image control console 310. The images obtained can be used by a physician to guide the guidewire 302 to a treatment site within the patient without the use of x-ray radiation.

As will be understood by those familiar with the art, the invention may be embodied in other specific forms without departing from the spirit or essential characteristics thereof. Likewise, the particular naming and division of the members, features, attributes, and other aspects are not mandatory or significant, and the mechanisms that implement the invention or its features may have different structural construct, names, and divisions. Accordingly, the disclosure of the invention is intended to be illustrative, but not limiting, of the scope of the invention.

While the invention has been described in terms of several embodiments, those of ordinary skill in the art will recognize that the invention is not limited to the embodiments described, but can be practiced with modification and alteration within the spirit and scope of the appended claims. The description is thus to be regarded as illustrative instead of limiting. There are numerous other variations to different aspects of the invention described above, which in the interest of conciseness have not been provided in detail. Accordingly, other embodiments are within the scope of the claims.

The invention has been described in relation to particular examples, which are intended in all respects to be illustrative rather than restrictive. Those skilled in the art will appreciate that many different combinations will be suitable for practicing the present invention. Other implementations of the invention will be apparent to those skilled in the art from consideration of the specification and practice of the invention disclosed herein. Various aspects and/or components of the described embodiments may be used singly or in any combination. It is intended that the specification and examples be considered as exemplary only, with a true scope and spirit of the invention being indicated by the following claims.

What is claimed is:

1. A system comprising:
a hypodermic needle body comprising a proximal end and a distal end and a plurality of surface artifacts in the body; and
an ultrasound transducer configured to emit ultrasound waveforms, wherein the surface artifacts are configured to reflect the ultrasound waveforms.

2. The system of claim 1, wherein the surface artifacts are selected from the group consisting of a double helix groove, dimples, pitts, and circular grooves.

3. A hypodermic needle comprising:
a needle tube main body comprising an edge at a tip end of the hypodermic needle, the needle tube having an outer surface;
at least a first helix groove on the outer surface of the needle tube beginning at a proximity of the tip end and spiraling at a first pitch; and
at least a second helix groove on the outer surface of the needle tube beginning at a proximity of the tip end and spiraling at a second pitch;
wherein the starting point of the first helix with respect of the tip end and the starting point of the second helix with respect of the tip end is different.

4. The hypodermic needle of claim 3, wherein the first starting point and the second starting point are opposite each other.

5. The hypodermic needle of claim 3, wherein the first pitch and the second pitch are equal.

6. The hypodermic needle of claim 3, wherein the second helix groove spirals counterclockwise.

7. The hypodermic needle claim 3, wherein the first helix groove and the second helix groove cross each other at at least one crossing point.

8. The hypodermic needle of claim 7, wherein the at least one crossing point is periodic.

9. A hypodermic needle providing for traceability at insertion using ultrasound, comprising:
a needle tube main body comprising an edge at a tip end of the hypodermic needle, the needle tube having an outer surface; and
a plurality of bands on the outer surface of the needle tube each of the plurality of bands having a length and each having a pattern that is at least different from an adjacent band;
wherein each pattern used for any band of the plurality of bands has a unique ultrasound deflection.

10. The hypodermic needle of claim 9, further comprising:
a patternless band, the patternless band positioned between two adjacent patterned bands of the plurality of bands.

11. The hypodermic needle of claim 9, further comprising:
a plurality of patternless bands, each of the plurality of patternless bands positioned between two adjacent patterned bands of the plurality of bands.

12. The hypodermic needle of claim 9, wherein the length of each of the plurality of bands is the same.

13. The hypodermic needle of claim 9, wherein each of the plurality of bands has a unique pattern.

14. The hypodermic needle of claim 9, wherein the pattern is selected from the group consisting of at least a double helix grooves, dimples, pitts, and circular grooves.

15. The hypodermic needle of claim 14, wherein a first helix of the at least a double helix has a first pitch and a second helix of the at least a double helix has a second pitch.

16. The hypodermic needle of claim 14, where the first helix spirals clockwise and the second helix spirals counterclockwise.

17. The hypodermic needle of claim 14, wherein the dimples are positioned randomly within a band.

18. The hypodermic needle of claim 14, wherein the dimples are positioned periodically within a band.

19. The hypodermic needle of claim 14, wherein the dimples are of: equal size or random size.

20. The hypodermic needle of claim 14, wherein the pitts are positioned randomly within a band.

21. The hypodermic needle of claim 14, wherein the pitts are positioned periodically within a band.

22. The hypodermic needle of claim 14, wherein the pitts are of: equal size or random size.
